(19) United States
(12) Patent Application Publication
(10) Pub. No.: US 2011/0280960 A1
(43) Pub. Date: Nov. 17, 2011

(54) THICK PINE NEEDLE EXTRACT COMPOSITION FOR CAPSULATION

(76) Inventors: Juris Rubens, Riga (LV); Irena Daberte, Riga (LV); Llze Barene, Riga (LV); Maris Daugavietis, Riga (LV)

(21) Appl. No.: 13/130,860

(22) PCT Filed: Dec. 4, 2008
(86) PCT No.: PCT/LV2009/000009

§ 371 Date: Jun. 21, 2011

(30) Foreign Application Priority Data
Dec. 4, 2008 (LV) P-08-25

(51) Int. Cl. A61K 36/13; A61P 29/00; A61P 39/06; A61P 17/00; A61K 33/30; A61P 1/00

(52) U.S. Cl. 424/641; 424/770

(57) ABSTRACT

The invention relates to pharmaceutics and to the pharmaceutical and food industries, in particular, to the production of compositions for stimulating the mucous membrane, ameliorating skin integument, and enhancing the organism's immunity. The aim of the invention is to develop a compound containing a thick pine needle extract with an improved quality composition in order to increase the biological activity of components and to preserve the processing characteristics and stability of the thick pine needle extract composition in capsular form. The aim is achieved by adding α-tocopherol (vitamin E), retinol (vitamin A), naphthoquinones (vitamin K), essential fatty acids (vitamin F) and microelements (zinc, selenium), with a specified component ratio, into the thick pine needle extract composition for capsulation.

### CROSS-REFERENCE TO RELATED APPLICATION

This application claims the benefit of the priority filing date in PCT/LV2009/000009 referenced in WIPO Publication WO 2010/064882 A1. The earliest priority date claimed is Dec. 4, 2008.

### FEDERALLY SPONSORED RESEARCH

None

### SEQUENCE LISTING OR PROGRAM

None

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention relates to pharmaceutics, parapharmaceutical preparations, and the food industry. It concerns issues of compositions that improve the state of the skin and the mucuous membranes, as well as enhancing the self-protective qualities of the human body. The goal of the invention is to develop a composition containing a highly dense and bioactive conifer needle extract with optimal technological qualities and a stable encapsulated form.

Various biologically active compositions containing vitamins and other bioactive components that stimulate the protective factors of the gastrointestinal tract are known to science [1-6].

Compositions prepared in the form of capsules have several advantages. They are easy to use, they contain the exact dosage, they lack a specific taste, and they are stable when stored [7-11].

Phitesten (dense conifer needle extract) is a complex medical preparation obtained from conifer needles and young conifer branches in full accordance with the Republic of Latvia Standard US-000312820-08-99 or ΓOCT 21769-84 [12]. This preparation is produced in the form of a dense paste-like mass. It has been recorded in the Latvian Medicinal Products List, No. 95-0002.

Dense conifer needle extract (DCNE) is a complex composition and a highly bioactive substance containing a great number of biologically active ingredients: carotenoids, polyprenols, chlorophyll acids, isoabienol, vitamins C, K and E, di- and triterpenoids, sescvyterpenoids, labdanoids, conifer wax, fungicides, fatty acids, stearins, and polymer compounds. The preparation has phytoantibiotic, antioxidant, biostimulating, and hepatoprotective qualities. It also serves as a protector of cell membranes and an immunomodulator [13-17]. DCNE and some of its ingredients—polyprenols and isoabienols—are widely used to improve gastrointestinal tract function and to increase indices of immunity, et al.

DCNE has a dense, thick consistency and a dark-green color. It has a specific odor and taste that somewhat complicates its direct administration and dosage. In order to eliminate these deficiencies, a special DCNE composition was developed specifically for encapsulation [18]. This DCNE composition is an actual prototype of the present invention.

To improve the quality of DCNE composition prior to encapsulation and to enhance its bioactive qualities, the composition should be supplemented with vitamins and microelements following a certain percentage pattern.

The present invention aims to develop a DCNE composition of improved quality and with enhanced biological activity of its components while retaining the technological qualities and stability of the DCNE composition in capsules.

### SUMMARY

The present invention aims to develop a Dense conifer needle extract (DCNE) composition of improved quality and with enhanced biological activity of its components while retaining the technological qualities and stability of the DCNE composition in capsular form. The aim is achieved by adding a-tocopherol (vitamin E), retinol (vitamin A), naphthoquinones (vitamin K), essential fatty acids (vitamin F) and microelements (zinc, selenium), with a specified component ratio, into the DCNE composition for capsulation.

### DESCRIPTION

The above described goal has been achieved by entering certain additional components into the composition—namely, alpha-tocopherol (vitamin E), retinol (vitamin A), naphthoquinones (vitamin K), and essential fatty acids (vitamin F) in the following ratio of components, mass %:

| | |
|---|---|
| Dense conifer needle extract | 20-70 |
| Emulsifier | 2-10 |
| Alpha-tocopherol (vitamin E) | 5-20 |
| Retinol (vitamin A) | 0.01-0.5 |
| Naphthoquinones (vitamin K) | 0.01-0.6 |
| Essential fatty acids (vitamin F) | 2-5 |
| Vegetable oil | the rest |

The composition may also contain certain microelements (zinc, selenium) at the following ratio of components, mass %:

| | |
|---|---|
| Dense conifer needle extract | 20-70 |
| Emulsifier | 2-10 |
| Alpha-tocopherol (vitamin E) | 5-20 |
| Retinol (vitamin A) | 0.01-0.5 |
| Naphthoquinones (vitamin K) | 0.01-0.6 |
| Essential fatty acids (vitamin F) | 2-5 |
| Microelements (zinc, selenium) | 0.01-0.1 |
| Vegetable oil | the rest |

Alpha-tocopherol (vitamin E) has antioxidant properties. It is a free radicals scavenger and therefore helps to prevent damage to cellular membranes. A decrease in alpha-tocopherol content may diminish the preparation's bioactive qualities and negatively affect the stability of the active components in DCNE.

Retinol (vitamin A) improves visual function and favourably affects the whole system. It contributes to a normalization of the epithelial tissue differentiation process and participates in regulating metabolism.

Naphthoquinones (vitamin K): A lack of vitamin K may affect the blood coagulation process. Naphthoquinones participate in the energy exchange process. They fulfill a significant coenzymatic function and participate in the synthesis of easy-to-restore proteins (digestive exoenzymes) and other enzymes.

Essential fatty acids (vitamin F) are administered to eliminate growth disorders, organism development disorders, skin function and dryness disorders, eczema backset, hair loss, nail brittleness and foliation, advanced gastrointestinal sensitivity to bacterial infections, pulmonary infections, decreased visual acuity, renal function disorders, etc.

Zinc (Zn) has the ability to neutralize free radicals, lower inflammation reactions, facilitate wound healing, and stabilize the activity of the skin's immune system.

Selenium (Se)—is a biologically active microelement that is included in several hormones and enzymes, and therefore linked with the functions of all organs, tissues and systems. Together with vitamin E, selenium supports vital activities of cells and prevents their necrosis. Thus, selenium is directly involved in processes affecting the youthfulness and longevity of the human body. Selenium is a strong antioxidant agent. It stimulates the growth of antibodies, and therefore enhances resistance to colds and infectious diseases. A lack of selenium may lead to inadequate immunity to colds and cancer.

When produced, DCNE is heated up to 45-50° C. Then, a solution of emulsifier and alpha-tocopherol (vitamin E) in oil is added while constantly mixing the substance until a homogenous mass appears. Also, retinol (vitamin A), naphthoquinones (vitamin K), essential fatty acids (vitamin F) and microelements (zinc and selenium) are added to the composition.

A composition containing DCNE, an emulsifier, vegetable oil and additional bioactive ingredients, and obtained in full compliance with the technology described in the invention, was successfully applied in the preparation of a dosed remedy in the form of soft gelatin capsules 0.3-1.0 g.

To prepare the above capsules, one needs the appropriate equipment capable of pressing capsules by a rotation-matrix method.

### EXAMPLE 1

When producing the composition, 20-70 g of DCNE is placed into a laboratory glass and heated in a water bath at 45-50° C. Separately, 2-10 g of an emulsifier (like glycerine monooleate), 5-20 g of alpha-tocopherol (vitamin E), 0.01-0.5 g of retinol (vitamin A), 0.01-0.6 g of naphthoquinones (vitamin K), 2-5 g of essential fatty acids (vitamin F) and vegetable oil are mixed and heated in a water bath of up to 45-50° C., and then poured into the DCNE.

### EXAMPLE 2

When producing the composition, 20-70 g of DCNE is placed into a laboratory glass and heated in a water bath at 45-50° C. Separately, 2-10 g of an emulsifier (such as glycerine monooleate), 5-20 g of alpha-tocopherol (vitamin E), 0.01-0.5 g of retinol (vitamin A), 0.01-0.6 g of naphthoquinones (vitamin K), 2-5 g of essential fatty acids (vitamin F), 0.01-0.1 g of microelements (zinc, selenium) and vegetable oil are mixed and heated in a water bath of up to 45-50° C., and then poured into the DCNE.

Each ingredient of the composition has specific, highly beneficial qualities. A combination of these qualities enables a synergetic effect that results in obtaining a DCNE based composition capable of encapsulation and rich with bioactive properties.

Decreasing the amount of DCNE to less than 20 g may reduce the bioactive qualities of the preparation; however, increasing the amount of DCNE over 70 g significantly complicates the technological process and storage of the preparation.

The emulsifier reduces the surface stretch on the borderline between the dispersion system phases, and supports the mixing of hydrophilic (DCNE) and lipophilic (vegetable oils, alpha-tocopherol, retinol etc.) components. Glycerin monooleate is applied as the emulsifier. Also, complex ethers of glycerin and the highest fatty acids, ethers of sorbitan and monoxyethylen sorbitan, distilled monoglycerides, natural phospholipids, and their combinations, may be used as emulsifiers. Decreasing the emulsifier content below 2 g or increasing its amount over 10 g complicates the technological process and impedes the procurement of a homogenous mixture.

Vegetable oil is necessary in procuring the optimal consistency of DCNE for insertion into soft gelatin caps.

Alpha-tocopherol (vitamin E) provides antioxidant qualities. When decreasing the amount of alpha-tocopherol below 5 g, the bioactivity of the preparation and the stability of the active ingredients of DCNE are reduced. Increasing alpha-tocopherol content over 20 g is not recommended.

Retinol (vitamin A) favourably affects the whole system. It may normalize the epithelial tissue differentiation process. When decreasing the amount of retinol below 0.01 g, the bioactive qualities of the preparation and the stability of the active ingredients of DCNE are reduced. Increasing the retinol content over 0.5 g is not recommended.

Experimental and clinical trials have demonstrated that DCNE stimulates the protective factors of gastric and duodenal mucosa. DCNE also has properties of an antioxidant scavenger, an antibacterial agent and an immunomodulator. It favourably affects the course of the inflammatory processes of gastric and duodenal mucosa.

The application of the vitamins and the microelements complex considerably enhances the biological and food value of DCNE, as well as the stability of its encapsulated form.

A composition containing DCNE, an emulsifier, vegetable oil and additional bioactive ingredients, and obtained in full compliance with the technology described in the invention, was successfully applied in the preparation of a dosed remedy in the form of soft gelatin capsules 0.3-1.0 g.

The amount of composition may be increased up to 10-100 kg, if needed, provided that the proportion of ingredients and the production technology are in full compliance with the invention description.

1. A composition of dense conifer needle extract ready for encapsulation containing an emulsifier and vegetable oil as well as additional ingredients—alpha-tocopherol (vitamin E), retinol (vitamin A), naphthoquinones (vitamin K) and essential fatty acids (vitamin F) in the following ratio of components, mass %:

| | |
|---|---|
| Dense conifer needle extract | 20-70 |
| Emulsifier | 2-10 |
| Alpha-tocopherol (vitamin E) | 5-20 |
| Retinol (vitamin A) | 0.01-0.5 |
| Naphthoquinones (vitamin K) | 0.01-0.6 |
| Essential fatty acids (vitamin F) | 2-5 |
| Vegetable oil | the rest |

2. A variation of the composition described in claim 1 also containing microelements (zinc, selenium) in the following ratio of components, mass %:

| | |
|---|---|
| Dense conifer needle extract | 20-70 |
| Emulsifier | 2-10 |
| Alpha-tocopherol (vitamin E) | 5-20 |
| Retinol (vitamin A) | 0.01-0.5 |
| Naphthoquinones (vitamin K) | 0.01-0.6 |
| Essential fatty acids (vitamin F) | 2-5 |
| Microelements (zinc, selenium) | 0.01-0.1 |
| Vegetable oil | the rest |
